(19) United States
(12) Patent Application Publication
(10) Pub. No.: US 2023/0248893 A1
(43) Pub. Date: Aug. 10, 2023

(54) SURGICAL DEVICES, SYSTEMS, AND METHODS FACILITATING MULTIPLE FLOW PATH FLUID MANAGEMENT

(71) Applicant: Medtronic Xomed, LLC, Jacksonville, FL (US)

(72) Inventors: Clint N.M. Lahey, Jacksonville, FL (US); Thoai D. Nguyen, Jacksonville, FL (US); Dale E. Slenker, Jacksonville, FL (US); Alexander J. Morley, Jacksonville, FL (US); Kathleen J. Bates, Boulder, CO (US); Ali Mowlai-Ashtiani, Jacksonville, FL (US); Keith W. Malang, Longmont, CO (US); Paul Hrenchir, Longmont, CO (US); Jonathan V. Hernandez, Jacksonville, FL (US); Manfred K. Luedi, Zwingen (CH)

(21) Appl. No.: 18/106,632

(22) Filed: Feb. 7, 2023

Related U.S. Application Data
(60) Provisional application No. 63/307,530, filed on Feb. 7, 2022.

(51) Int. Cl. A61M 1/00

(52) U.S. Cl. CPC A61M 1/72; A61M 1/65; A61M 1/782

(57) ABSTRACT

A fluid management cassette includes a cassette body configured for insertion into a cassette bay of a control console, the cassette body including a face plate having at least one fluid input port and at least first and second fluid output ports defined therethrough. A pump is disposed within the cassette body as is a valve manifold, first and second pinch valves, or at least one pressure sensitive valve. First and second fluid flow paths extending through the cassette body have shared connections to the pump and separate connections to the respective first and second fluid output ports. The valve manifold, first and second pinch valves, or at least one pressure sensitive valve is selectively actuatable to at least four different configurations to permit fluid flow along either, neither, or both of the first and second fluid flow paths.

### CROSS REFERENCE TO RELATED APPLICATION

This application claims the benefit of and priority to U.S. Provisional Pat. Application No. 63/307,530, filed on Feb. 7, 2022, the entire contents of which is incorporated by reference herein.

### FIELD

The present disclosure relates to surgical fluid management and, more specifically, to surgical devices, systems, and methods facilitating multiple flow path fluid management.

### BACKGROUND

Various surgical devices and systems utilize fluid management to facilitate performing a surgical task such as, for example, to irrigate a treatment site, aspirate a treatment site, clean a surgical device, wash a treatment site, clear a field of view, cool a surgical device, etc. Some nonlimiting examples of these types of surgical devices include micro-debriders, surgical drills, suction-irrigators, tissue shavers, endoscopes, balloon or other catheters, energy-based devices, and the like.

Some surgical devices require fluid management of multiple fluid flow paths, for example, to enable two or more of irrigation, aspiration, cleaning, lavage, clearing, cooling, etc. As the number of fluid flow paths increases, additional tubing and/or valve structures are required to enable selective and independent management of each fluid flow path. This challenge is complicated by the desire to avoid contamination of capital equipment with fluid, e.g., avoiding fluid contact with the fluid management console and, instead, confining fluid travel to within the disposable, e.g., the cassette connectable to the fluid management console.

### SUMMARY

The terms “about,” substantially,” and the like, as utilized herein, are meant to account for manufacturing, material, environmental, use, and/or measurement tolerances and variations, and in any event may encompass differences of up to 10%. Further, to the extent consistent, any of the aspects described herein may be used in conjunction with any or all of the other aspects described herein.

Provided in accordance with aspects of the present disclosure is a fluid management cassette including a cassette body configured for insertion into a cassette bay of a control console. The cassette body includes a face plate having at least one fluid input port and at least first and second fluid output port defined therethrough. The cassette further includes a pump disposed within the interior of the cassette body, a valve manifold disposed within an interior of the cassette body, and first and second fluid flow paths extending through the cassette body. The first and second fluid flow paths share a connection from the first fluid input port to the pump and from the pump to the valve manifold. The first and second fluid flow paths define separate connections from the valve manifold to the respective first and second fluid output ports. The valve manifold is selectively actuatable to at least four different configurations including a first configuration wherein fluid flow along both the first and second fluid flow paths to the respective first and second fluid output ports is inhibited, a second configuration wherein fluid flow along the first fluid flow path to the first fluid output port is permitted and fluid flow along the second fluid flow path to the second fluid output port is inhibited, a third configuration wherein fluid flow along the first fluid flow path to the first fluid output port is inhibited and fluid flow along the second fluid flow path to the second fluid output port is permitted, and a fourth configuration wherein fluid flow along both the first and second fluid flow paths to the respective first and second fluid output ports is permitted.

In an aspect of the present disclosure, the valve manifold includes a base and a slider translationally slidable within the base to different positions to achieve the first, second, third, and fourth configurations.

In another aspect of the present disclosure, a spring biases the slider towards a position corresponding to one of the first, second, third, or fourth configurations.

In another aspect of the present disclosure, the slider is configured for translational sliding in response to translational actuation of an external motor driven actuator.

In still another aspect of the present disclosure, the valve manifold includes a base and a rotary valve rotatably disposed within the base and rotatable to different positions to achieve the first, second, third, and fourth configurations.

In yet another aspect of the present disclosure, the rotary valve is configured for rotation in response to rotational actuation of an external motor driven actuator.

In still yet another aspect of the present disclosure, the pump is configured for actuation in response to rotational actuation of an external motor driven actuator.

Another fluid management cassette provided in accordance with aspects of the present disclosure includes a cassette body configured for insertion into a cassette bay of a control console, the cassette body including a face plate defining at least one fluid input port and at least first and second fluid output ports. The cassette further includes a pump disposed within an interior of the cassette body and first and second pinch valves disposed within the cassette body. First and second fluid flow paths extend through the cassette body, share a connection from the first fluid input port to the pump, and define separate first and second output connections, respectively, from the pump to the respective first and second fluid output ports, The first and second pinch valves are operably coupled to the respective first and second output connections and are selectively and independently actuatable in response to actuation of first and second external actuators, respectively, to achieve at least four different configurations including a first configuration wherein fluid flow along both the first and second fluid flow paths to the respective first and second fluid output ports is inhibited, a second configuration wherein fluid flow along the first fluid flow path to the first fluid output port is permitted and fluid flow along the second fluid flow path to the second fluid output port is inhibited, a third configuration wherein fluid flow along the first fluid flow path to the first fluid output port is inhibited and fluid flow along the second fluid flow path to the second fluid output port is permitted, and a fourth configuration wherein fluid flow along both the first and second fluid flow paths to the respective first and second fluid output ports is permitted.

In an aspect of the present disclosure, the first and second pinch valves are selectively and independently actuatable in response to actuation of first and second motor-driven actuators, respectively.

In another aspect of the present disclosure, the first and second pinch valves are selectively and independently actuatable in response to actuation of first and second motor driven translational actuators, respectively.

In still another aspect of the present disclosure, the first and second pinch valves are selectively and independently actuatable in response to actuation of first and second electric actuators, respectively.

In yet another aspect of the present disclosure, actuation of each of the first and second electric actuators is configured to generate one of an electric field or a magnetic field to actuate the respective first and second pinch valves.

In still yet another aspect of the present disclosure, the pump is configured for actuation in response to rotational actuation of an external motor driven actuator.

Another fluid management cassette provided in accordance with aspects of the present disclosure includes a cassette body configured for insertion into a cassette bay of a control console, the cassette body including a face plate having at least one fluid input port and at least first and second fluid output ports defined therethrough. The cassette further includes a pump disposed within the cassette body and at least one pressure sensitive valve disposed within the cassette body. First and second fluid flow paths extend through the cassette body sharing a connection from the first fluid input port to the pump, sharing or defining separate connections from the pump to the at least one pressure sensitive valve, and defining separate connections from the at least one pressure sensitive valve to the first and second fluid output ports, respectively. The at least one pressure sensitive valve is disposed between the pump and the first and second fluid output ports and is selectively actuatable in response to pulses of fluid pressure provided by the pump to achieve at least four different configurations including a first configuration wherein fluid flow along both the first and second fluid flow paths to the respective first and second fluid output ports is inhibited, a second configuration wherein fluid flow along the first fluid flow path to the first fluid output port is permitted and fluid flow along the second fluid flow path to the second fluid output port is inhibited, a third configuration wherein fluid flow along the first fluid flow path to the first fluid output port is inhibited and fluid flow along the second fluid flow path to the second fluid output port is permitted, and a fourth configuration wherein fluid flow along both the first and second fluid flow paths to the respective first and second fluid output ports is permitted.

In an aspect of the present disclosure, the pump is configured for actuation in response to rotational actuation of an external motor driven actuator.

In another aspect of the present disclosure, the at least one pressure sensitive valve includes first and second pressure sensitive valves wherein the first pressure sensitive valve is disposed between the pump and the first fluid output port and the second pressure sensitive valve is disposed between the pump and the second fluid output port.

In yet another aspect of the present disclosure, the first and second fluid flow paths define separate connections from the pump to the first and second pressure sensitive valves.

In still another aspect of the present disclosure, the at least one pressure sensitive valve includes a single pressure sensitive valve disposed between the pump and the first and second fluid output ports.

In still yet another aspect of the present disclosure, the first and second fluid flow paths define a shared connection from the pump to the single pressure sensitive valve.

In another aspect of the present disclosure, the single pressure sensitive valve has at least first, second, third, and fourth positions corresponding to the respective at least first, second, third, and fourth configurations.

### BRIEF DESCRIPTION OF THE DRAWINGS

Various aspects and features of the present disclosure are described hereinbelow with reference to the drawings wherein:

FIG. 1 is a perspective view of a surgical system provided in accordance with aspects of the present disclosure;

FIG. 2A is a schematic illustration of a fluid management cassette in accordance with the present disclosure configured for use with the surgical system of FIG. 1 and operably engaged within a control console of the surgical system of FIG. 1 in a first fluid flow configuration;

FIGS. 2B-2D are partial schematic illustrations of the fluid management cassette of FIG. 2A operably engaged within the control console of FIG. 1 in various other fluid flow configurations;

FIG. 3A is a schematic illustration of another fluid management cassette in accordance with the present disclosure configured for use with the surgical system of FIG. 1 and operably engaged within the control console of the surgical system of FIG. 1 in a first fluid flow configuration;

FIGS. 3B-3D are partial schematic illustrations of the fluid management cassette of FIG. 3A operably engaged within the control console of FIG. 1 in various other fluid flow configurations;

FIG. 4 is a schematic illustration of another fluid management cassette configured for use with the surgical system of FIG. 1 operably engaged within the control console of the surgical system of FIG. 1;

FIG. 5 is a schematic illustration of still another fluid management cassette configured for use with the surgical system of FIG. 1 operably engaged within the control console of the surgical system of FIG. 1;

FIG. 6 is a schematic illustration of yet another fluid management cassette configured for use with the surgical system of FIG. 1 operably engaged within the control console of the surgical system of FIG. 1; and

FIG. 7 is a schematic illustration of still yet another fluid management cassette configured for use with the surgical system of FIG. 1 operably engaged within the control console of the surgical system of FIG. 1.

### DETAILED DESCRIPTION

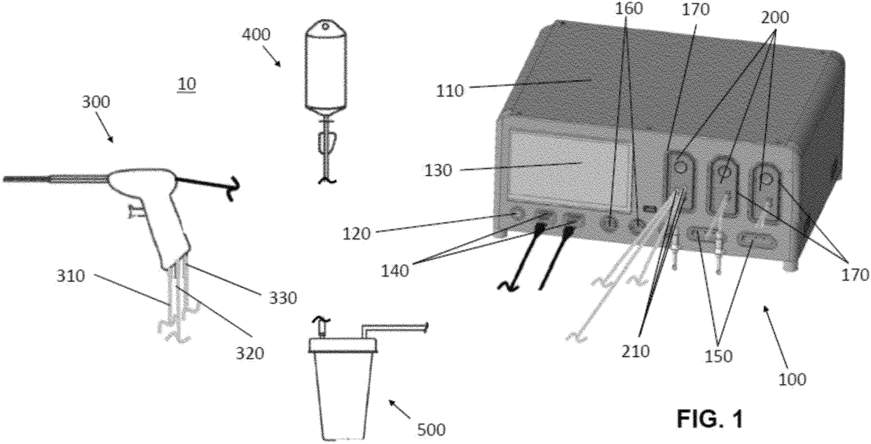

Referring to FIG. 1, a surgical system 10 provided in accordance with the present disclosure generally includes: a control console 100; one or more fluid management cassettes 200 configured for releasable engagement with control console 100; one or more surgical instruments 300 configured to be powered, controlled, energized, supplied fluid, and/or supplied vacuum by control console 100; one or more fluid sources 400; and/or one or more fluid collection canisters 500. Although plural fluid management cassettes 200, surgical instruments 300, fluid sources 400, and fluid collection canisters 500 are contemplated, surgical system 10 is described below with reference to only one fluid management cassette 200, one surgical instrument 300, one fluid source 400, and one fluid collection canister 500 for the purposes of brevity and understanding. Likewise, although control console 100 may include plural similar components, each of such may be described in the singular hereinbelow for the purposes of brevity and understanding.

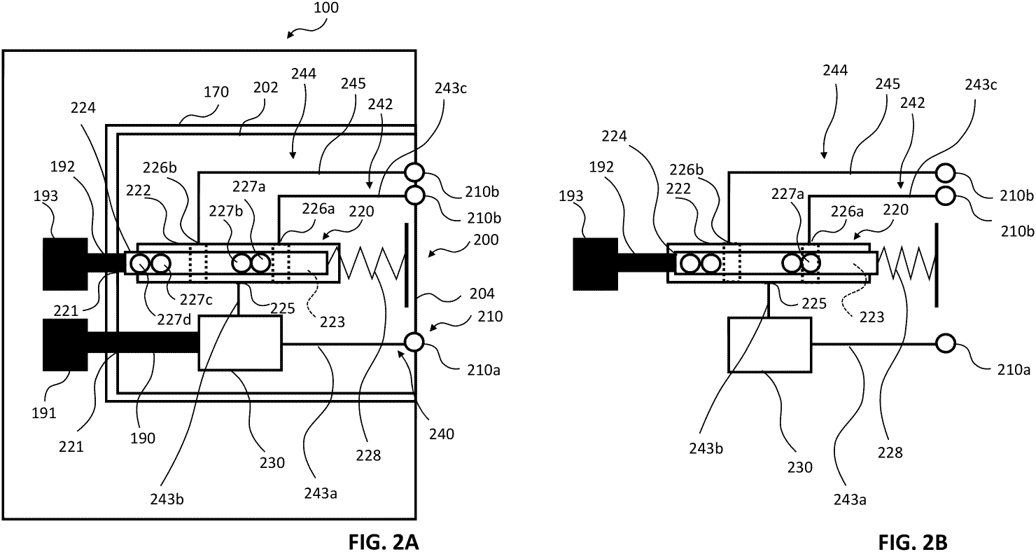

Control console 100 includes: a housing 110, a power button 120; a graphical user interface 130, one or more power ports 140 for powering and controlling connected powered surgical instrument(s), one or more energy ports 150 for providing surgical energy, e.g., nonpolar, bipolar, microwave, ultrasonic, thermal, light, and/or other surgical energy, to connected energy-based surgical instrument(s), one or more additional ports 160; and a plurality of cassette bays 170. Control console 100 further includes one or more central processing units (CPU’s) and/or microcontroller units (MCU’s), power generating and control hardware, surgical energy generating and control hardware, and any other suitable hardware and corresponding firmware/software stored thereon for operating and controlling operation of surgical instruments 300 connected thereto. Control console 100 further includes one or more actuators 190, 192, 194a, 194b (FIGS. 2A-7) operably positioned relative to a corresponding cassette bay 170 to control the various fluid paths 240 (FIGS. 2A-2D) defined through fluid management cassette 200 when fluid management cassette 200 is received within cassette bay 170. One of the CPU’s and/or MCU’s may also control the one or more actuators 190, 192, 194a, 194b (FIGS. 2A-7) according to a particular control program selected (e.g., via graphical user interface 130), according to user actuation of one or more controls associated with surgical instrument 300, in response to sensed feedback, and/or in any other suitable manner.

Fluid management cassette 200, as noted above, is receivable within cassette bay 170 to operably couple the fluid flow paths 240 (FIGS. 2A-2D) defined therethrough with the corresponding one or more actuators 190, 192, 194a, 194b (FIGS. 2A-7) to enable selective control of the various fluid flow paths 240 (FIGS. 2A-2D), e.g., to close, open, and/or control fluid flow along the various fluid flow paths 240 (FIGS. 2A-2D). Various configurations of fluid management cassettes (see FIGS. 2A-7) suitable for use as fluid management cassette 200 in accordance with the present disclosure are described in greater detail below, as are corresponding actuators 190, 192, 194a, 194b (FIGS. 2A-7) configured to selectively drive and control the fluid flow through the cassettes. Regardless of its particular configuration, fluid management cassette 200 may include a plurality of external tubing ports 210 to enable connection of suitable tubing to fluidly couple fluid management cassette 200 with one or more fluid flow paths associated with surgical instrument 300, fluid source 400, and/or fluid collection canister 500.

Continuing with reference to FIG. 1, surgical instrument 300, as noted above, may be powered, controlled, energized, supplied fluid, and/or supplied vacuum by control console 100. Surgical instrument 300 may be configured as, for example and without limitation, one or more of a micro-debrider, surgical drill, suction-irrigator, tissue shaver, endoscope, sheath for an endoscope (e.g., a lens-cleaning sheath), balloon or other catheter, energy-based device, fluid-cooled device, etc. In some aspects, surgical instrument 300 defines at least three fluid flow paths 310, 320, 330 therethrough or otherwise associated therewith such as, for example, an irrigation fluid flow path 310, a lavage fluid flow path 320, and a suction fluid flow path 330.

Fluid source 400, e.g., an IV-style fluid bag, is fluidly coupled to one or more fluid flow paths defined within fluid management cassette 200. Fluid management cassette 200, as noted above, is connected to surgical instrument 300 and is configured to enable control console 100 to control the delivery of fluid from fluid source 400 to the surgical instrument 300. More specifically, as an example, fluid management cassette 200 may operably couple fluid source 400 with irrigation fluid flow path 310 and lavage fluid flow path 320 of surgical instrument 300 to enable control console 100 to selectively supply fluid along fluid flow paths 310, 320.

Fluid collection canister 500 is fluidly coupled between the surgical instrument 300 and fluid management cassette 200 to enable control console 100 to control the suctioning of fluid from the surgical instrument 300 into the fluid collection canister 500, e.g., along suction fluid flow path 330.

Turning to FIGS. 2A-2D, fluid management cassette 200 in accordance with aspects of the present disclosure is shown operably engaged with a cassette bay 170 of control console 100. Fluid management cassette 200 includes a cassette body 202 defining a face plate 204 (formed with or attached to on cassette body 202) that defines the plurality of external tubing ports 210 (FIG. 1), e.g., one or more input tubing ports 210a and a plurality of output tubing ports 210b, that enable connection of fluid management cassette 200 with fluid source 400 (FIG. 1), surgical instrument 300 (FIG. 1), and/or fluid collection canister 500 (FIG. 1). Cassette body 202 encloses the internal components and features of fluid management cassette 200 while allowing selective access thereto, as detailed below. More specifically, cassette body 202 defines a plurality of access openings 221 to enable actuators 190, 192, e.g., output shafts of motors 191, 193 to extend into cassette body 202 when fluid management cassette 200 is operably engaged within cassette bay 170 such that actuators 190, 192 can be selectively actuated to open, close, and/or otherwise control the fluid flow paths 240 defined through fluid management cassette 200, as detailed below.

Continuing with reference to FIGS. 2A-2D, fluid management cassette 200 includes a valve manifold 220 disposed within cassette body 202, a pump 230 disposed within cassette body 202, and, as noted above, includes a plurality of external tubing ports 210 including one or more input tubing ports 210a and a plurality of output tubing ports 210b. Each of the plurality of fluid flow paths 240 defined through fluid management cassette 200 is operably coupled to one or more input tubing ports 210a and one or more output tubing ports 210b. At least one of the fluid flow paths 240, more specifically, is operably coupled to pump 230 within fluid management cassette 200 and at least one of the fluid flow paths 240 (which may be the same or a different fluid flow path 240) is operably coupled to valve manifold 220 within fluid management cassette 200. Fluid management cassette 200 may include one or more fluid flow paths 240 operably coupled to fluid pump 230 and valve manifold 220 and/or one or more fluid flow paths 240 that are separate from both fluid pump 230 and valve manifold 220. Further still, at least two of the fluid flow paths 240 defined through fluid management cassette 200 may have shared inputs or outputs such as, for example, where a single fluid source is connected to one of the input tubing ports 210a and wherein the fluid flow path 240 splits within fluid management cassette 200 to provide two selectively controllable outputs at two of the output tubing ports 210b. Any suitable combination of fluid flow paths 240 are detailed above may be provided.

Fluid management cassette 200 is shown including a first fluid flow path 242 and a second fluid flow path 244. First fluid flow path 242 includes tubing 243a connecting one of the input tubing ports 210a to an input of pump 230 and tubing 243b connects an output of pump 230 with an input 225 to valve manifold 220. Further, tubing 243c of first fluid flow path 242 connects a first output 226a of valve manifold 220 with one of the output tubing ports 210b.

Second fluid flow path 244 shares tubing 243a and tubing 243b with first flow path 244 and, thus, also extends through valve manifold 220 and pump 230. Second fluid flow path 244 further includes tubing 245 that connects a second output 226b of valve manifold 220 with another one of the output tubing ports 210b. Fluid source 400 (FIG. 1), or any other suitable fluid source, can be connected to the input tubing port 210a associated with tubing 243a to thus supply fluid for selective pumping along fluid flow path 242 and/or fluid flow path 244 and out the corresponding output tubing port 210b.

Pump 230 is disposed between shared tubing 243a and shared tubing 243b of fluid flow paths 242, 244. Pump 230 is configured to be driven by a first actuator 190 of control console 100. More specifically, upon insertion of fluid management cassette 200 into cassette bay 170 of control console 100, first actuator 190 extends into cassette body 202 to operably engage pump 230. First actuator 190 may be an output shaft of motor 191 which may be, for example, a rotary output motor configured to rotationally drive first actuator 190 to thereby activate pump 230. Motor 191 may be configured to drive first actuator 190 at a single, constant speed and, thus, pump 230 may be operated in an ON/OFF manner. Alternatively, motor 191 may be configured to drive first actuator 190 at a plurality of different speed settings and, thus, pump 230 may be configured to operate at a plurality of different settings such as, for example, high, medium, and low. In aspects, motor 191 is controllable to thereby drive pump 230 to achieve a desired fluid flow pressure or fluid flow rate, e.g., based on feedback from flow pressure and/or flow rate sensors (not shown).

Regardless of the particular configuration of motor 191 and the manner in which pump 230 is operated, pump 230, as noted above, is disposed between shared tubing 243a and shared tubing 243b of fluid flow paths 242, 244. Thus, pump 230 itself is incapable of independently pumping fluid along tubing 243c for output from the output port 210b associated with first fluid flow path 242 and along tubing 245 for output from the output port 210b associated with second fluid flow path 244. Valve manifold 220, disposed between shared tubing 243b and separate tubing 243c, 245 of first and second fluid flow paths 242, 244, respectively, enables the output selection of either, both, or neither of first and second fluid flow paths 242, 244, thus enabling selective fluid flow control along first and second fluid flow paths 242, 244 of fluid management cassette 200 with a single pump 230.

Referring still to FIGS. 2A-2D, valve manifold 220 includes a base 222 defining an internal chamber 223, and a slider 224 slidable within internal chamber 223 of base 222 in sealed relation therewith. Base 222 further defines an input 225 disposed in fluid communication with internal chamber 223 and first and second outputs 226a, 226b disposed in fluid communication with internal chamber 223. Input 225 is configured to fluidly connect to shared tubing 243b while first and second outputs 226a, 226b are configured to fluidly connect to tubing 243c and tubing 245, respectively.

Slider 224 defines a plurality of lumens 227a, 227b, 227c, 227d defined transversely therethrough and arranged in a pre-determined pattern such that, as slider 224 is slid to different positions within internal chamber 223, fluid communication between input 225 and first and/or second outputs 226a, 226b is selectively established. Slider 224 may be biased towards an initial position via a spring 228. Slider 224 is further configured to be driven by a second actuator 192 of control console 100. More specifically, upon insertion of fluid management cassette 200 into cassette bay 170 of control console 100, second actuator 192 extends into cassette body 202 to operably engage slider 224 of valve manifold 220. Second actuator 192 may be an output shaft of motor 193 which may be, for example, a solenoid, servo or other suitable displacement output motor configured to translationally drive second actuator 192 to thereby urge slider 224 through base 222 against the bias of spring 228 or to enable slider 224 to return in the opposite direction under the bias of spring 228.

Slider 224, more specifically, is drivable, e.g., via second actuator 192 and motor 193, to a plurality of different positions to thereby establish or cut off a fluid flow path from pump 230 to tubing 243c for output from the output port 210b associated with first fluid flow path 242 and/or along tubing 245 for output from the output port 210b associated with second fluid flow path 244. More specifically, in a first position of slider 224, as shown in FIG. 2A and which may correspond to an initial or home position of slider 224, none of the lumens 227a, 227b, 227c, 227d of slider 224 establish fluid communication between input 225 and first or second outputs 226a, 226b. Thus, in this position, valve manifold 220 inhibits fluid flow therethrough.

In a second position of slider 224, as shown in FIG. 2B, lumen 227a of slider 224 defines a fluid flow path from input 225 to first output 226a; however, no connection is made from input 225 to second output 226b. Thus, in this second position, valve manifold 220 permits fluid flow for output to tubing 243c and, thus, for output from the output port 210b associated with first fluid flow path 242. However, in this second position, output along the second fluid flow path 244 is still inhibited.

In a third position of slider 224, as shown in FIG. 2C, lumen 227b of slider 224 defines a fluid flow path from input 225 to first output 226a; and lumen 227c of slider 224 defines a fluid flow path from input 225 to second output 226b. Thus, in this third position, valve manifold 220 permits fluid flow for output to tubing 243c and, thus, for output from the output port 210b associated with first fluid flow path 242, as well as to tubing 245 and, thus, for output from the output port 210b associated with second fluid flow path 244.

In a fourth position of slider 224, as shown in FIG. 2D, lumen 227d of slider 224 defines a fluid flow path from input 225 to second output 226b; however, no connection is made from input 225 to first output 226a. Thus, in this fourth position, valve manifold 220 permits fluid flow for output to tubing 245 and, thus, for output from the output port 210b associated with second fluid flow path 244. However, in this fourth position, output along the first fluid flow path 242 is still inhibited.

As demonstrated above, valve manifold 220 of fluid management cassette 200, as controlled via second actuator 192 of control console 100, enables fluid to be selectively pumped from a single pump 230 and with shared tubing 243a, 243b, out either, both, or neither of first and second fluid flow paths 242, 244.

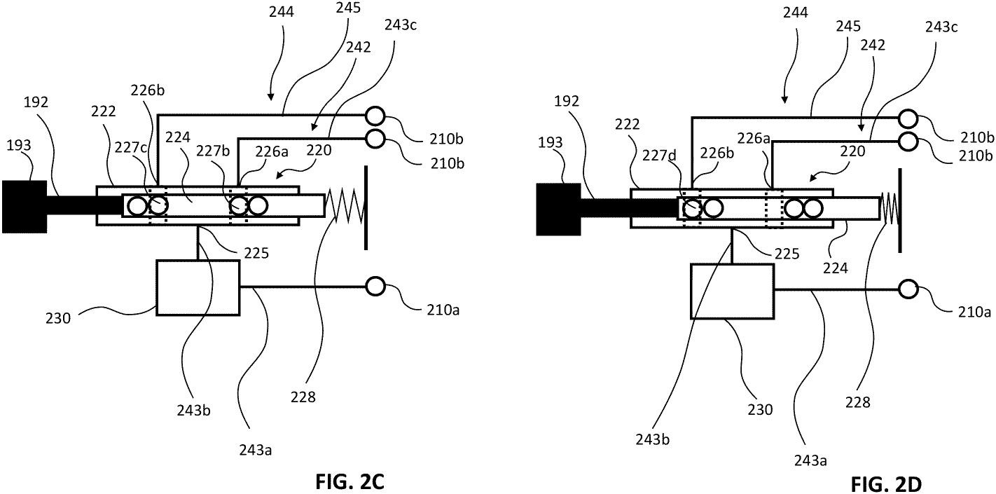

Turning to FIGS. 3A-3D, another fluid management cassette 1200 in accordance with aspects of the present disclosure is shown operably engaged with a cassette bay 170 of control console 100. Fluid management cassette 1200 is similar to and may include any of the features of fluid management cassette 200 (FIGS. 2A-2D) detailed above except as explicitly contradicted below. Accordingly, only differences between fluid management cassette 1200 and fluid management cassette 200 (FIGS. 2A-2D) are described in detail below while similarities are summarily described or omitted entirely.

Fluid management cassette 1200 includes a valve manifold 1220 disposed within cassette body 1202, a pump 1230 disposed within cassette body 1202, and first and second fluid flow paths 1242, 1244. First and second fluid flow paths 1242, 1244 share tubing 1243a from a common input tubing ports 1210a to an input of pump 1230 and tubing 1243b which connects an output of pump 1230 with an input 1225 to valve manifold 1220. Further, tubing 1243c of first fluid flow path 1242 connects a first output 1226a of valve manifold 1220 with one of the output tubing ports 1210b while tubing 1245 of second fluid flow path 1244 connects a second output 1226b of valve manifold 1220 with another one of the output tubing ports 1210b.

Valve manifold 1220 includes a base 1222 defining an internal chamber 1223, and a rotary valve 1224 rotatable within internal chamber 1223 of base 1222 in sealed relation therewith. Base 1222 further defines the input 1225 disposed in fluid communication with internal chamber 1223 and first and second outputs 1226a, 1226b disposed in fluid communication with internal chamber 1223.

Rotary valve 1224 is configured to be driven by a second actuator 1192 of control console 100. More specifically, upon insertion of fluid management cassette 1200 into cassette bay 170 of control console 100, second actuator 1192 extends into cassette body 1202 to operably engage rotary valve 1224 of valve manifold 1220. Second actuator 1192 may be an output shaft of motor 1193 which may be, for example, a rotary output motor configured to rotationally drive second actuator 1192 to thereby rotate rotary valve 1224 within base 1222. Alternatively, gearing, linkages, or other suitable components may be utilized to convert translational motion from a translational second actuator into rotary motion of rotary valve 1224.

In a first position of rotary valve 1224, as shown in FIG. 3A, fluid communication between input 1225 and both first or second outputs 1226a, 1226b is inhibited. Thus, in this first position, valve manifold 1220 inhibits fluid flow therethrough.

In a second position of rotary valve 1224, as shown in FIG. 3B, rotary valve 1224 is positioned to allow fluid flow from input 1225 to first output 1226a but to inhibit fluid flow from input 1225 to second output 1226b. Thus, in this second position, valve manifold 1220 permits fluid flow for output along the first fluid flow path 1242 but not the second fluid flow path 1244.

In a third position of rotary valve 1224, as shown in FIG. 3C, rotary valve 1224 is positioned to allow fluid flow from input 1225 to both first output 1226a and second output 1226b. Thus, in this third position, valve manifold 1220 permits fluid flow for output along first fluid flow path 1242, as well as second fluid flow path 1244.

In a fourth position of rotary valve 1224, as shown in FIG. 3D, rotary valve 1224 is positioned to inhibit fluid flow from input 1225 to first output 1226a and to allow fluid flow from input 1225 to second output 1226b. Thus, in this fourth position, valve manifold 1220 inhibits fluid flow for output along the first fluid flow path 1242 while allowing fluid flow for output along the second fluid flow path 1244.

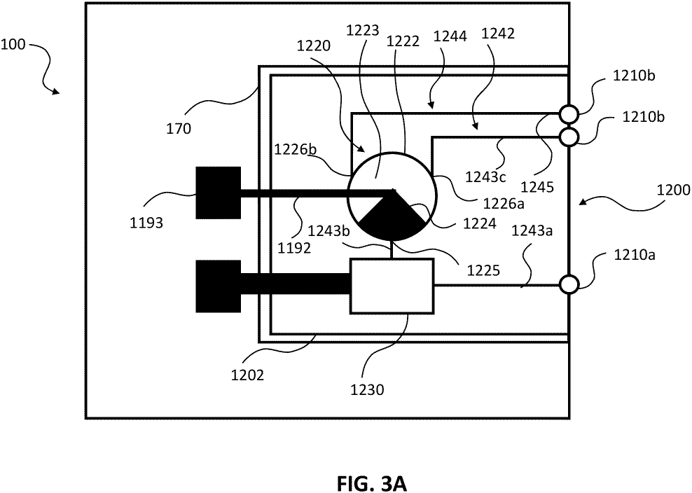

With reference to FIG. 4, another fluid management cassette 2200 in accordance with aspects of the present disclosure is shown operably engaged with a cassette bay 170 of control console 100. Fluid management cassette 2200 is similar to and may include any of the features of fluid management cassette 200 (FIGS. 2A-2D) detailed above except as explicitly contradicted below. Accordingly, only differences between fluid management cassette 2200 and fluid management cassette 200 (FIGS. 2A-2D) are described in detail below while similarities are summarily described or omitted entirely.

Fluid management cassette 2200 includes first and second pinch valves 2220a, 2220b, respectively, disposed within cassette body 1202, a pump 2230 disposed within cassette body 2202, and first and second fluid flow paths 2242, 2244. First and second fluid flow paths 2242, 2244 share tubing 2243a from a common input tubing ports 2210a to an input of pump 2230. Tubing 2243b of first fluid flow path 2242 connects a first output of pump 2230 with one of the output tubing ports 2210b while tubing 2245 of second fluid flow path 2244 connects a second output of pump 2230 with another one of the output tubing ports 2210b.

First and second pinch valves 2220a, 2220b are operably coupled to tubing 2243b, 2245, respectively. First and second pinch valves 2220a, 2220b are configured to be driven by third actuators 194a, 194b, respectively, of control console 100. More specifically, upon insertion of fluid management cassette 2200 into cassette bay 170 of control console 100, third actuators 194a, 194b extend into cassette body 2202 to operably engage pinch valves 2220a, 2220b, respectively (either directly or indirectly via intervening structures). Third actuators 194a, 194b may be output shafts of motors 195a, 195b, which may be solenoid motors, servo motors, or other suitable displacement output motors configured to translationally drive third actuators 194a, 194b to thereby actuate first and second pinch valves 2220a, 2220b to selectively pinch closed tubing 2243b of first fluid flow path 2242 and tubing 2245 of second fluid flow path 2244, respectively, to inhibit the flow of fluid therethrough. Thus, first and second pinch valves 2220a, 2220b, as controlled third actuators 194a, 194b of control console 100, enable fluid to be selectively pumped from a single pump 2230 and with shared tubing 2243a, out either, both, or neither of first and second fluid flow paths 2242, 2244.

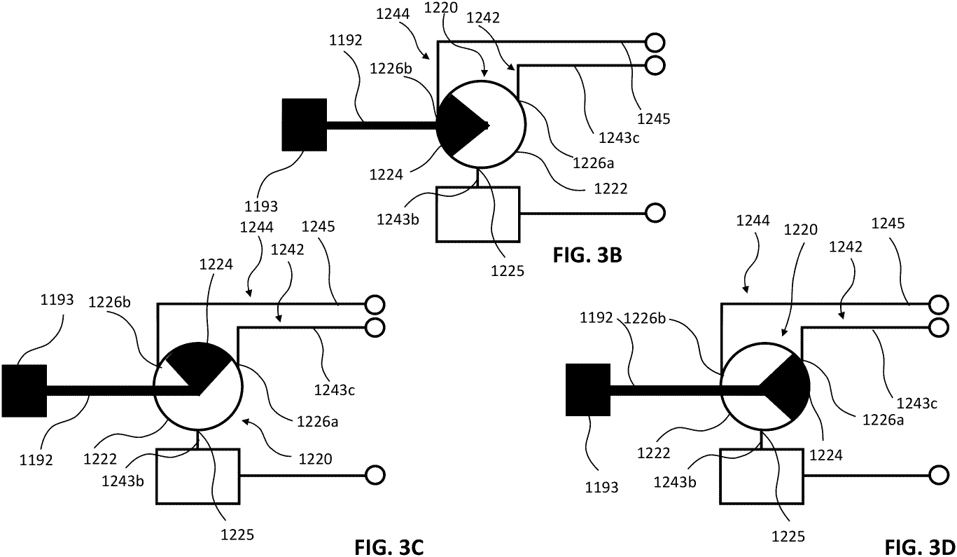

FIG. 5 illustrates still another fluid management cassette 3200 in accordance with aspects of the present disclosure. Fluid management cassette 3200 is similar to fluid management cassette 2200 (FIG. 4) and may include any of the features thereof. Fluid management cassette 3200 differs from fluid management cassette 2200 (FIG. 4) in that rather than mechanically activated pinch valves (e.g., activated via translating actuators), fluid management cassette 3200 includes electronic pinch valves 3220a, 3220b. More specifically, upon insertion of fluid management cassette 3200 into cassette bay 170 of control console 100, first and second electronic actuators 196a, 196b, e.g., electrical and/or magnetic field or signal generators, are operably positioned relative to electronic pinch valves 3220a, 3220b such that, upon energization of first and/or second electronic actuators 196a, 196b, e.g., by generating an electrical and/or magnetic field or signal, the corresponding electronic pinch valve(s) 3220a, 3220b is/are activated to pinch closed first fluid flow path 3242 and/or second fluid flow path 3244, respectively.

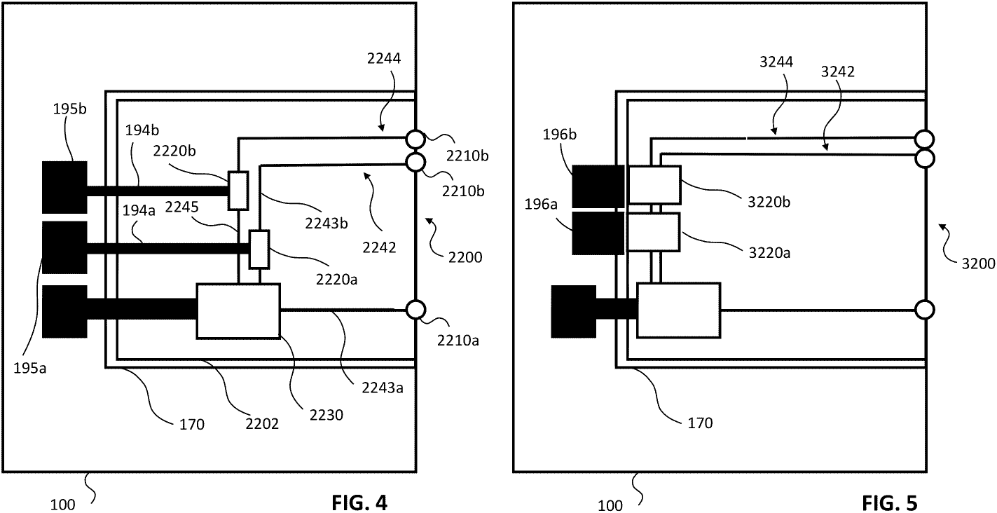

Referring to FIG. 6, yet another fluid management cassette 4200 in accordance with aspects of the present disclosure is shown operably engaged with a cassette bay 170 of control console 100. Fluid management cassette 4200 is similar to fluid management cassette 2200 (FIG. 4) and may include any of the features thereof; thus, only differences therebetween are described in detail while similarities are summarily described or omitted entirely.

Fluid management cassette 4200 includes first and second pressure sensitive valves 4220a, 4220b disposed within cassette body 4202, a pump 4230 disposed within cassette body 4202, and first and second fluid flow paths 4242, 4244. First and second fluid flow paths 4242, 4244 share tubing 4243a from a common input tubing ports 4210a to an input of pump 4230. The output tubing from pump 4230 splits into tubing 4243b of first fluid flow path 4242 which connects with one of the output tubing ports 4210b and tubing 4245 of second fluid flow path 4244 which connects a second output of pump 4230 with another one of the output tubing ports 4210b.

Rather than requiring an additional actuator to control the valves 4220a, 4220b, first and second pressure sensitive valves 4220a, 4220b are controlled via pump 4230 which, in turn, is controlled via actuator 190 and motor 191 of control console 100. More specifically, pump 4230 is controlled via actuator 190 and motor 191 to provide pressurized pulses of fluid to first and second pressure sensitive valves 4220a, 4220b in order to selectively open and close first and second pressure sensitive valves 4220a, 4220b. The open and close thresholds for first and second pressure sensitive valves 4220a, 4220b may be different such that pump 4230 can provide one or more pressurized pulses of fluid to achieve at least four different fluid flow configurations: a first configuration wherein both first and second pressure sensitive valves 4220a, 4220b are closed inhibiting fluid flow along first and second fluid flow paths 4242, 4244, respectively; a second configuration wherein first pressure sensitive valve 4220a is open but second pressure sensitive valve 4220b is closed thus allowing fluid flow only along first fluid flow path 4242; a third configuration wherein first pressure sensitive valve 4220a is closed and second pressure sensitive valve 4220b is open thus allowing fluid flow only along second fluid flow path 4244; and a fourth configuration wherein both first and second pressure sensitive valves 4220a, 4220b are open allowing fluid flow along first and second fluid flow paths 4242, 4244, respectively.

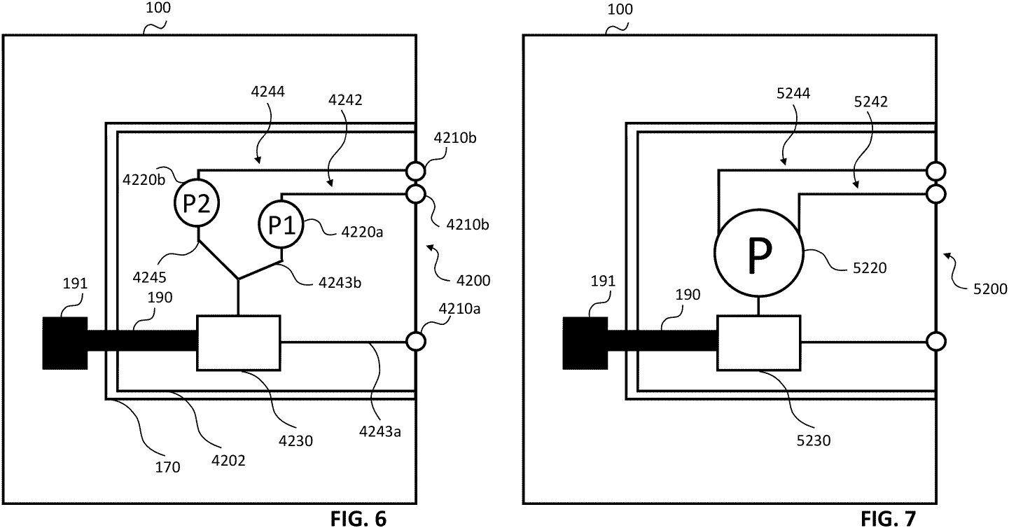

Turning to FIG. 7, still yet another fluid management cassette 5200 in accordance with aspects of the present disclosure is shown similar to fluid management cassette 4200 (FIG. 6) and including any of the features thereof except as explicitly contradicted below. Fluid management cassette 5200, more specifically, differs from fluid management cassette 4200 (FIG. 6) in that rather than providing first and second ON/OFF pressure sensitive valves, fluid management cassette 5200 includes a single, at least four position pressure sensitive valve 5220.

Pump 5230 of fluid management cassette 5200 is controlled via actuator 190 and motor 191 to provide pressurized pulses of fluid to pressure sensitive valve 5220 in order to selectively manipulate pressure sensitive valve 5220 to one of the at least four positions: a first position corresponding to a first configuration wherein fluid flow along both first and second fluid flow paths 5242, 5244, respectively, is inhibited; a second position corresponding to a second configuration wherein fluid flow is only permitted along first fluid flow path 5242; a third position corresponding to a third configuration wherein fluid flow is only permitted along second fluid flow path 5244; and a fourth position corresponding to configuration wherein fluid flow along both first and second fluid flow paths 5242, 5244, respectively, is permitted.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A fluid management cassette, comprising:
a cassette body configured for insertion into a cassette bay of a control console, the cassette body including a face plate having at least one fluid input port and at least first and second fluid output ports defined therethrough;
a pump disposed within an interior of the cassette body;
a valve manifold disposed within the interior of the cassette body; and
first and second fluid flow paths extending through the cassette body, the first and second fluid flow paths sharing connection from the first fluid input port to the pump and from the pump to the valve manifold, the first and second fluid flow paths defining separate connections from the valve manifold to the respective first and second fluid output ports,
wherein the valve manifold is selectively actuatable to at least four different configurations including a first configuration wherein fluid flow along both the first and second fluid flow paths to the respective first and second fluid output ports is inhibited, a second configuration wherein fluid flow along the first fluid flow path to the first fluid output port is permitted and fluid flow along the second fluid flow path to the second fluid output port is inhibited, a third configuration wherein fluid flow along the first fluid flow path to the first fluid output port is inhibited and fluid flow along the second fluid flow path to the second fluid output port is permitted, and a fourth configuration wherein fluid flow along both the first and second fluid flow paths to the respective first and second fluid output ports is permitted.

2. The fluid management cassette according to claim 1, wherein the valve manifold includes a base and a slider translationally slidable within the base to different positions to achieve the first, second, third, and fourth configurations.

3. The fluid management cassette according to claim 2, further comprising a spring biasing the slider towards a position corresponding to one of the first, second, third, or fourth configurations.

4. The fluid management cassette according to claim 2, wherein the slider is configured for translational sliding in response to translational actuation of an external motor driven actuator.

5. The fluid management cassette according to claim 1, wherein the valve manifold includes a base and a rotary valve rotatably disposed within the base and rotatable to different positions to achieve the first, second, third, and fourth configurations.

6. The fluid management cassette according to claim 5, wherein the rotary valve is configured for rotation in response to rotational actuation of an external motor driven actuator.

7. The fluid management cassette according to claim 1, wherein the pump is configured for actuation in response to rotational actuation of an external motor driven actuator.

8. A fluid management cassette, comprising:
a cassette body configured for insertion into a cassette bay of a control console, the cassette body including a face plate having at least one fluid input port and at least first and second fluid output ports defined therethrough;
a pump disposed within an interior of the cassette body;
first and second pinch valves disposed within the int interior of cassette body; and
first and second fluid flow paths extending through the cassette body, the first and second fluid flow paths sharing connection from the first fluid input port to the pump, the first and second fluid flow paths defining separate first and second output connections, respectively, from the pump to the respective first and second fluid output ports, the first and second pinch valves operably coupled to the respective first and second output connections,
wherein the first and second pinch valves are selectively and independently actuatable in response to actuation of first and second external actuators, respectively, to achieve at least four different configurations including a first configuration wherein fluid flow along both the first and second fluid flow paths to the respective first and second fluid output ports is inhibited, a second configuration wherein fluid flow along the first fluid flow path to the first fluid output port is permitted and fluid flow along the second fluid flow path to the second fluid output port is inhibited, a third configuration wherein fluid flow along the first fluid flow path to the first fluid output port is inhibited and fluid flow along the second fluid flow path to the second fluid output port is permitted, and a fourth configuration wherein fluid flow along both the first and second fluid flow paths to the respective first and second fluid output ports is permitted.

9. The fluid management cassette according to claim 8, wherein the first and second pinch valves are selectively and independently actuatable in response to actuation of first and second motor-driven actuators, respectively.

10. The fluid management cassette according to claim 9, wherein the first and second pinch valves are selectively and independently actuatable in response to actuation of first and second motor driven translational actuators, respectively.

11. The fluid management cassette according to claim 8, wherein the first and second pinch valves are selectively and independently actuatable in response to actuation of first and second electric actuators, respectively.

12. The fluid management cassette according to claim 11, wherein actuation of each of the first and second electric actuators is configured to generate one of an electric field or a magnetic field to actuate the respective first and second pinch valves.

13. The fluid management cassette according to claim 8, wherein the pump is configured for actuation in response to rotational actuation of an external motor driven actuator.

14. A fluid management cassette, comprising:
a cassette body configured for insertion into a cassette bay of a control console, the cassette body including a face plate having at least one fluid input port and at least first and second fluid output ports defined therethrough;
a pump disposed within an interior of the cassette body;
at least one pressure sensitive valve disposed within the interior of the cassette body; and
first and second fluid flow paths extending through the cassette body, the first and second fluid flow paths sharing connection from the first fluid input port to the pump, defining separate or shared connections from the pump to the at least one pressure sensitive valve, and defining separate connections from the at least one pressure sensitive valve to the first and second fluid output ports, respectively,
wherein the at least one pressure sensitive valve is disposed between the pump and the first and second fluid output ports, the at least one pressure sensitive valve selectively actuatable in response to pulses of fluid pressure provided by the pump to achieve at least four different configurations including a first configuration wherein fluid flow along both the first and second fluid flow paths to the respective first and second fluid output ports is inhibited, a second configuration wherein fluid flow along the first fluid flow path to the first fluid output port is permitted and fluid flow along the second fluid flow path to the second fluid output port is inhibited, a third configuration wherein fluid flow along the first fluid flow path to the first fluid output port is inhibited and fluid flow along the second fluid flow path to the second fluid output port is permitted, and a fourth configuration wherein fluid flow along both the first and second fluid flow paths to the respective first and second fluid output ports is permitted.

15. The fluid management cassette according to claim 14, wherein the pump is configured for actuation in response to rotational actuation of an external motor driven actuator.

16. The fluid management cassette according to claim 14, wherein the at least one pressure sensitive valve includes first and second pressure sensitive valves, the first pressure sensitive valve disposed between the pump and the first fluid output port and the second pressure sensitive valve disposed between the pump and the second fluid output port.

17. The fluid management cassette according to claim 16, wherein the first and second fluid flow paths define separate connections from the pump to the first and second pressure sensitive valves.

18. The fluid management cassette according to claim 14, wherein the at least one pressure sensitive valve includes a single pressure sensitive valve disposed between the pump and the first and second fluid output ports.

19. The fluid management cassette according to claim 18, wherein the first and second fluid flow paths define a shared connection from the pump to the single pressure sensitive valve.

20. The fluid management cassette according to claim 18, wherein the single pressure sensitive valve has at least first, second, third, and fourth positions corresponding to the respective at least first, second, third, and fourth configurations.
